(19) United States
(12) Patent Application Publication
(10) Pub. No.: US 2021/0060335 A1
(43) Pub. Date: Mar. 4, 2021

(54) Massaging Tool for Orbicularis Oculi Muscle

(71) Applicant: Lucinni Bailar, Houston, TX (US)

(72) Inventor: Lucinni Bailar, Houston, TX (US)

(21) Appl. No.: 16/558,572

(22) Filed: Sep. 3, 2019

(51) Int. Cl. A61N 1/36

(52) U.S. Cl. CPC A61N 1/36003; A61N 1/3603

(57) ABSTRACT

A massaging tool for orbicularis oculi muscle includes an elongated handle, a control panel, an electrical-pulse discharging electrode, an electric muscle stimulation (EMS) unit, and a power source. The control panel, which is completed with a power switch and at least one intensity control button, is integrated onto the elongated handle. The electrical-pulse discharging electrode is adjacently connected to the elongated handle. The EMS unit that continuously generates a timely released electrical-pulse is mounted within the elongated handle. The power source is mounted within the elongated handle and electrically connected to the EMS unit through the control panel. The EMS unit is electrically connected to the electrical-pulse discharging electrode so that the electrical-pulse discharging electrode can apply the timely released electrical-pulse to the outer layer of skin around the orbicularis oculi muscle.

### FIELD OF THE INVENTION

The present invention generally relates to facial massaging tool. More specifically, the present invention is a massaging tool that utilizes Electrical Muscle Stimulation (EMS) technology so that the EMS technology can flex the orbicularis oculi muscle and increase muscle mass to prevent upper eyelid dropping and lower eyelid sagging by firming up the orbicularis oculi muscle.

### BACKGROUND OF THE INVENTION

At-home spa products allows many individuals to improve skincare and overall wellbeing at home and at a lower cost. One of the most common type and favored at-home spa products focus on facial skincare. This is because aging, regardless if an individual is 25 or 50, reduces the firmness of the skin. The outer layer of skin receives less blood flow and nutrients from the lower layer of skin of the face. Gaps between the outer layer of skin and the lower layer of skin increase. The prevention and reduction of appearance of aging requires direct action against the outer layer of skin.

Electrical Muscle Stimulation (EMS), also known as Neuromuscular Electrical Stimulation (NMES), is the elicitation of muscle contraction using electric impulses and is utilized to exercises specific muscles that are difficult to activate. More specifically, EMS unit sends small and precisely timed electrical pulses to the nerve supplying the target muscle. These electrical pulses act as a message, telling the muscle fibers to ‘contract’. The muscle fibers contract in much the same way as if the message had come from the brain. In other words, these electrical pulses are able to activate the muscles via the nerves.

It is therefore an objective of the present invention to combine a facial massaging tool and an EMS unit so that present invention is able to stimulate blood flow and the delivery of nutrients between the lower layer of skin and the outer layer of skin of the face. The present invention accommodates varying types of skin and maximizes the stimulation of the outer layer of skin with respect to the orbicularis oculi muscle around the eye. The present invention is a portable device that allows a user to easily maneuver around the eye. Moreover, the present invention effectively encourages oxygen/blood flow to flow through the outer layer of skin of the face to increase collagen and tone muscles.

### BRIEF DESCRIPTION OF THE DRAWINGS

FIG. 1 is a front view of the present invention, showing the first half.

FIG. 2 is a side view of the present invention, wherein the dash lines illustrate the EMS unit and the power source within the elongated handle.

FIG. 3 is a bottom view of the present invention, showing the charging port that is associated with the rechargeable battery.

FIG. 4 is a rear view of the present invention, showing the removable cover that is associated with the primary battery.

FIG. 5 is a schematic view showing the electrical connection of the present invention with respect to the rechargeable battery.

FIG. 6 is a schematic view showing the electrical connection of the present invention with respect to the primary battery.

### DETAIL DESCRIPTIONS OF THE INVENTION

All illustrations of the drawings are for the purpose of describing selected versions of the present invention and are not intended to limit the scope of the present invention.

The present invention is a facial massaging tool that massages the outer layer of skin into the lower layer of skin of the face via electric muscle stimulation technology. The present invention is specifically designed to increase the blood flow to the orbicularis oculi muscle around the eye. Resultantly, the blood flow is able to tone muscles and increase collagen around the eye area thus minimizing the skin sagging and aging appearance.

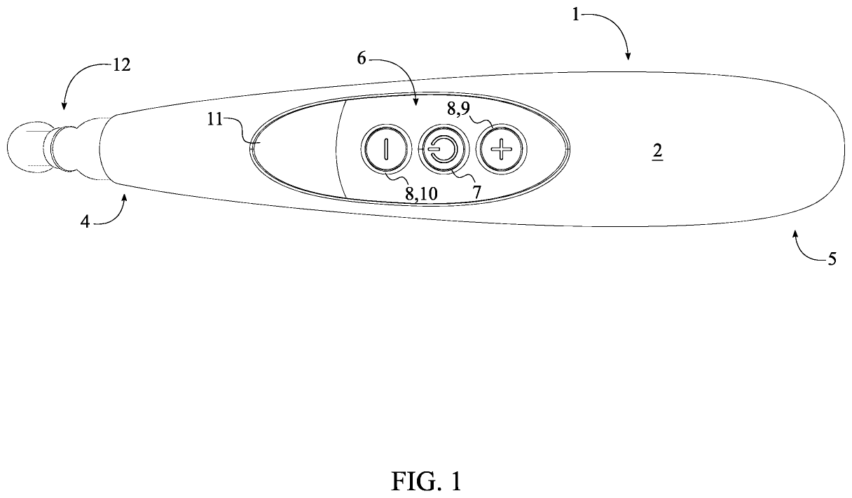

The present invention comprises an elongated handle 1, a control panel 6, an electrical-pulse discharging electrode 12, an electric muscle stimulation (EMS) unit 13, and a power source 14 as shown in FIG. 1-6. In reference to the general configuration of the present invention, the control panel 6 that enables the manual operation of the present invention is integrated onto the elongated handle 1. The user can easy grasp and maneuver the present invention by the elongated handle 1 as the electrical-pulse discharging electrode 12 is adjacently connected to the elongated handle 1. The EMS unit 13 generates small and timely released electric pulses so that the electrical-pulse discharging electrode 12 can discharge them onto the user's orbicularis oculi muscle. More specifically, the EMS unit 13 can flex the orbicularis oculi muscle and increase muscle mass to prevent upper eyelid dropping and lower eyelid sagging by firming up the orbicularis oculi muscle. To maintain an obstruction free outer surface for the elongated handle 1 and to improve the reliability of the present invention, the EMS unit 13 and the power source 14 are mounted within the elongated handle 1. In order to activate/power the present invention, the power source 14 is electrically connected to the EMS unit 13 through the control panel 6 while the EMS unit 13 is electrically connected to the electrical-pulse discharging electrode 12.

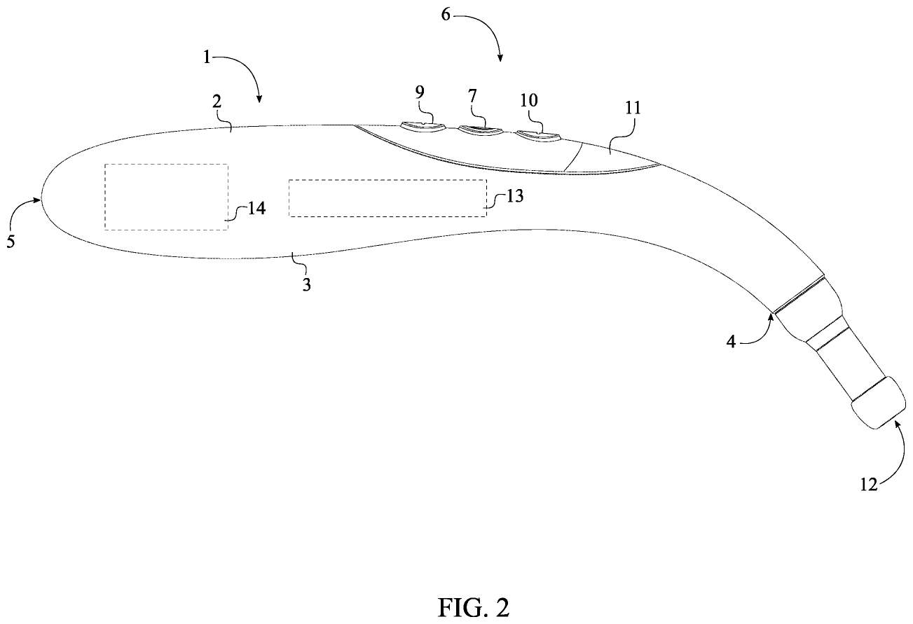

The elongated handle 1 also functions as the main structural body within the present invention in addition to allowing a surface area to grasp and maneuver the present invention. In reference to FIG. 1-2, the elongated handle 1 comprises a first half 2, a second half 3, a top end 4, and a bottom end 5. More specifically, the first half 2 being extended from the top end 4 to the bottom end 5, and the second half 3 being extended from the top end 4 to the bottom end 5. The first half 2 and the second half 3 are perimetrically mounted to each other thus completing the structural body of the elongated handle 1 so that the control panel 6 and the electrical-pulse discharging electrode 12 can be externally integrated onto the elongated handle 1. Furthermore, the first half 2 and the second half 3 delineates a hollow body so that the EMS unit 13 and the power source 14 can be internally integrated into the elongated handle 1.

The control panel 6 that manually operates the present invention comprises a power switch 7, at least one intensity control button 8, and a display panel 11 as shown in FIG. 1-2. The power switch 7 is able to turn-on and turn-off the EMS unit 13 as the power source 14 is selectively connected or disconnected via the power switch 7. The power switch 7 is laterally connected onto the first half 2 of the elongated handle 1 and positioned in between the top end 4 and the bottom end 5 of the elongated handle 1. The display panel 11 is connected onto the first half 2 of the elongated handle 1 and positioned adjacent to the top end 4. As a result, the display panel 11 is able to display certain functionalities of the present invention. For example, the display panel 11 can display different intensity levels of the EMS unit 13, a low battery notification, date and time, and other related notifications/information. The at least one intensity control button 8 is laterally connected onto the first half 2 of the elongated handle 1 and positioned adjacent to the power switch 7. Resultantly, the at least one intensity control button 8 is able to manually control the intensity of the EMS unit 13. Furthermore, the power switch 7 and the at least one intensity control button 8 are laterally connected to the elongated handle 1 in such way that the positioning of the power switch 7 and the at least one intensity control button 8 are adjacent to the thumb of the user's grip thus facilitating easy access.

In reference to FIG. 1-2, the at least one intensity control button 8 preferably comprises an intensity increasing button 9 and an intensity decreasing button 10. More specifically, the intensity increasing button 9 and the intensity decreasing button 10 are oppositely positioned of each other about the power switch 7. The intensity increasing button 9 is positioned in between the power switch 7 and the bottom end 5. The intensity decreasing button 10 is positioned in between the power switch 7 and the top end 4. When the present invention is powered through the power switch 7, the lowest intensity level that is set as the default starting intensity is activated within the present invention. Then, the user can press the intensity increasing button 9 to increases the intensity levels of the EMS unit 13 until the EMS unit 13 reaches the highest intensity level. When the intensity levels of the EMS unit 13 need to be lowered, the user can simply press on the intensity decreasing button 10 until the EMS unit 13 reaches the lowest intensity level which is also the default starting intensity. In order to easily identify each of the intensity levels of the EMS unit 13, each intensity level is designated with a numerical value as the corresponding numerical value is displayed through the display panel 11.

In some embodiment of the present invention, the at least one intensity control button 8 can be a single button that incrementally changes the intensity levels of the EMS unit 13 as cycle of intensity levels. For example, when the present invention is powered through the power switch 7, the lowest intensity level that is set as the default starting intensity is activated within the present invention. Then, the user can press the intensity increasing button 9 to increases the intensity levels of the EMS unit 13 until the EMS unit 13 reaches the highest intensity level. Once the highest intensity level is reached within the present invention, the operation of the intensity increasing button 9 cycles back to the lowest intensity level which is also the default starting intensity. In order to easily identify each of the intensity levels of the EMS unit 13, each intensity level is designated with a numerical value as the corresponding numerical value is displayed through the display panel 11.

The electrical-pulse discharging electrode 12 that discharges the small and timely released electric pulses onto the user's orbicularis oculi muscle functions as the contact point between the present invention and the user's skin. The electrical-pulse discharging electrode 12 is configured reach the user's orbicularis oculi muscle through an upright grasping of the elongated handle 1, as shown in FIG. 2, the electrical-pulse discharging electrode 12 is adjacently connected to the top end 4 of the elongated handle 1 thus allowing the user to easily secure the present invention with the elongated handle 1. Furthermore, the electrical-pulse discharging electrode 12 sends the small and timely released electric pulses to the nerve supplying the orbicularis oculi muscle. These small and timely released electric pulses act as a message, telling the muscle fibers to contract as if the message had come from the brain.

The EMS unit 13 that generates small and timely released electric pulses is fully enclosed by the first half 2 and the second of the elongated handle 1 so that the EMS unit 13 can be protected from outside elements. As mentioned before, the EMS unit 13 is equipped with different intensity levels which can be selected through the at least one intensity control button 8.

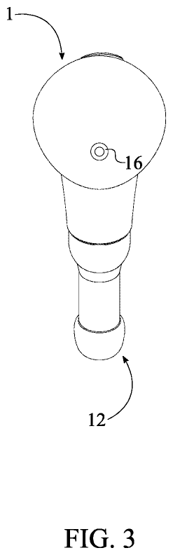

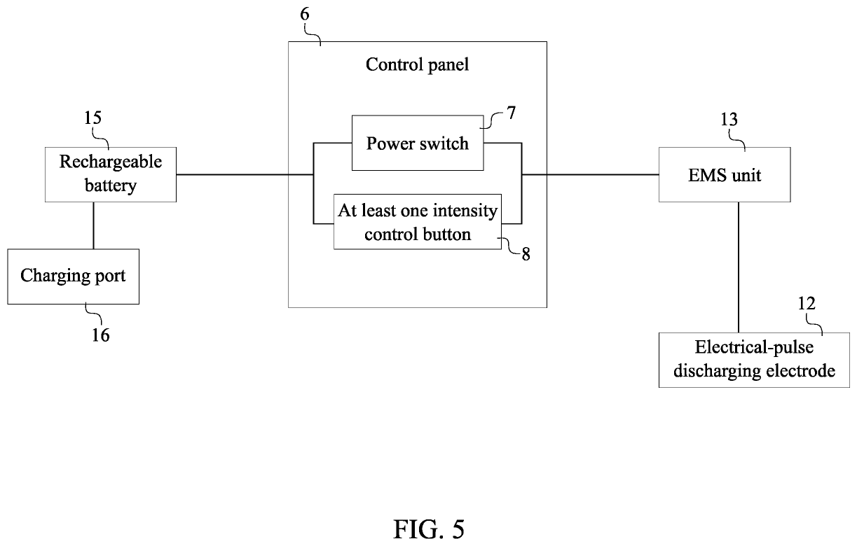

In reference to FIG. 3 and FIG. 5, the power source 14 comprises a rechargeable battery 15 and a charging port 16. More specifically, the rechargeable battery 15 is mounted within the first half 2 and the second half 3 of the elongated handle 1 so that the EMS unit 13 and the control panel 6 can be electrically powered. The charging port 16 traverses through the elongated handle 1 and electrically connected to the rechargeable battery 15. Resultantly, the charging port 16 is able to recharge the rechargeable battery 15 through an extension cord that electrically connects with the charging port 16 when the rechargeable battery 15 is drained.

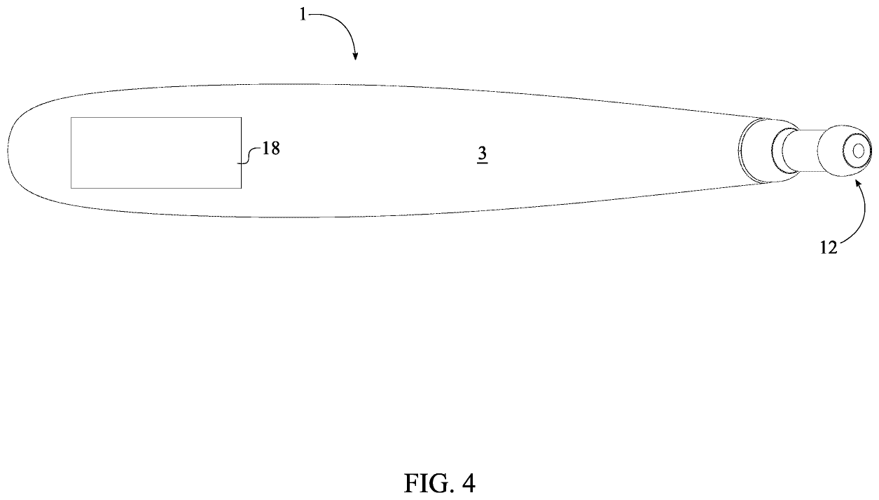

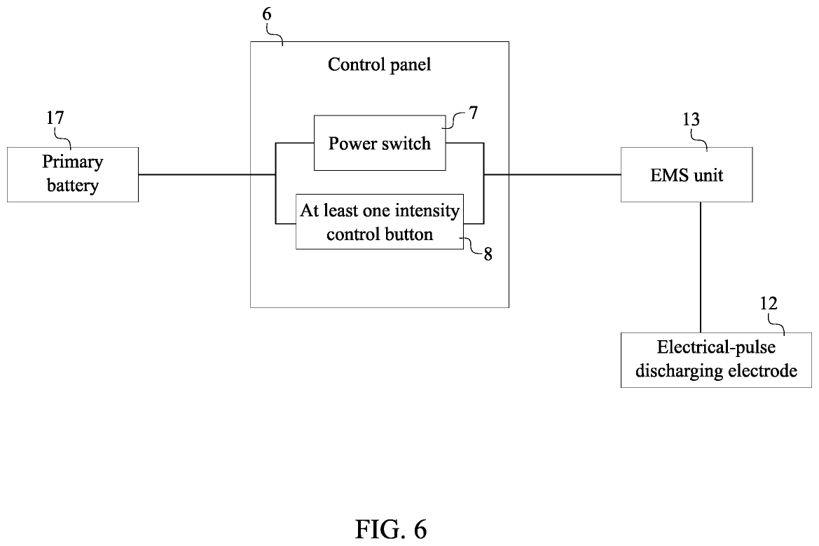

In reference to FIG. 4 and FIG. 6, the power source 14 comprises a primary battery 17 and a removable cover 18. More specifically, the primary battery 17 is mounted within the first half 2 and the second half 3 of the elongated handle 1 so that the EMS unit 13 and the control panel 6 can be electrically powered. The removable cover 18 is integrated onto the second half 3 of the elongated handle 1 and encloses the primary battery 17. Resultantly, the user can access and change the primary battery 17 through the removable cover 18 when the primary battery 17 is drained.

Although the invention has been explained in relation to its preferred embodiment, it is to be understood that many other possible modifications and variations can be made without departing from the spirit and scope of the invention as hereinafter claimed.

What is claimed is:

1. A massaging tool for orbicularis oculi muscle comprising:
an elongated handle;
a control panel;
an electrical-pulse discharging electrode;
an electric muscle stimulation (EMS) unit;
a power source;
the control panel being integrated onto the elongated handle;
the electrical-pulse discharging electrode being adjacently connected to the elongated handle;
the EMS unit being mounted within the elongated handle;
the power source being mounted within the elongated handle;
the power source being electrically connected to the EMS unit through the control panel; and
the EMS unit being electrically connected to the electrical-pulse discharging electrode, wherein the EMS unit flexes the orbicularis oculi muscle and increases muscle mass to prevent upper eyelid dropping and lower eyelid sagging by firming up the orbicularis oculi muscle.

2. The massaging tool for orbicularis oculi muscle as claimed in claim 1 comprising:
the control panel comprising a power switch and at least one intensity control button;
the power switch being laterally connected onto a first half of the elongated handle;
the power switch being positioned in between a top end and a bottom end of the elongated handle;
the at least one intensity control button being laterally connected onto the first half of the elongated handle; and
the at least one intensity control button being positioned adjacent to the power switch.

3. The massaging tool for orbicularis oculi muscle as claimed in claim 2 comprising:
the elongated handle further comprising a second half;
the first half being extended from the top end to the bottom end;
the second half being extended from the top end to the bottom end; and
the first half and the second half being perimetrically mounted to each other.

4. The massaging tool for orbicularis oculi muscle as claimed in claim 2 comprising:
the at least one intensity control button comprising an intensity increasing button and an intensity decreasing button;
the intensity increasing button and the intensity decreasing button being oppositely positioned of each other about the power switch;
the intensity increasing button being positioned in between the power switch and the bottom end; and
the intensity decreasing button being positioned in between the power switch and the top end.

5. The massaging tool for orbicularis oculi muscle as claimed in claim 1 comprising:
the control panel further comprising a display panel;
the display panel being connected onto a first half of the elongated handle; and
the display panel being positioned adjacent to the top end.

6. The massaging tool for orbicularis oculi muscle as claimed in claim 1, wherein the electrical-pulse discharging electrode is adjacently connected to a top end of the elongated handle.

7. The massaging tool for orbicularis oculi muscle as claimed in claim 1, wherein the EMS unit is enclosed by a first half and a second half of the elongated handle.

8. The massaging tool for orbicularis oculi muscle as claimed in claim 1 comprising:
the power source comprising a rechargeable battery and a charging port;
the rechargeable battery being mounted within a first half and a second half of the elongated handle;
the charging port traversing through the elongated handle; and
the charging port being electrically connected to the rechargeable battery.
